(19) United States
(12) Patent Application Publication
(10) Pub. No.: US 2014/0271723 A1
(43) Pub. Date: Sep. 18, 2014

(54) ADJUVANT COMPOSITIONS AND METHODS OF USING THEREOF

(71) Applicants: Saint Louis University, St. Louis, MO (US); Auxagen, Inc., St. Louis, MO (US)

(72) Inventors: Shuan Shian Huang, St. Louis, MO (US); Jung San Huang, St. Louis, MO (US)

(21) Appl. No.: 14/209,344

(22) Filed: Mar. 13, 2014

Related U.S. Application Data
(60) Provisional application No. 61/786,832, filed on Mar. 15, 2013.

(51) Int. Cl. A61K 39/39

(52) U.S. Cl. CPC A61K 39/39
USPC 424/277.1; 530/324; 424/278.1; 530/327; 530/326; 530/328; 530/329

(57) ABSTRACT

The present invention relates to adjuvant compositions, vaccine compositions, and methods of enhancing an immune response to an antigen.

### CROSS REFERENCE TO RELATED APPLICATIONS

This application claims the priority of U.S. provisional application 61/786,832, filed Mar. 15, 2013, which is hereby incorporated by reference in its entirety.

### GOVERNMENTAL RIGHTS

This invention was made with government support under Grant Nos. AR 52578 and AA 019223 awarded by the National Institutes of Health (NIH). The government has certain rights in the invention.

### FIELD OF THE INVENTION

The present invention relates to adjuvant compositions, vaccine compositions, and methods of enhancing an immune response to an antigen.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821(f).

### BACKGROUND OF THE INVENTION

Adjuvants are agents that are not immunogenic themselves, but augment the immune response when used in combination with an antigen in a vaccine composition. Currently, the most commonly used adjuvants in humans are aluminum hydroxide, aluminum phosphate and calcium phosphate. Due to safety and toxicity concerns, only aluminum hydroxide, or alum, has been the only approved adjuvant for human use in the United States. However, alum is comparatively weak and will only enhance the immune response when used with certain disease vaccines. In addition, the alum adjuvant has very limited value in the development of many potential vaccines against viruses or tumors.

Accordingly, an urgent need exists for effective adjuvants, especially adjuvants for vaccines against viruses or tumors.

### BRIEF DESCRIPTION OF THE DRAWINGS

The application file contains at least one photograph executed in color. Copies of this patent application publication with color photographs will be provided by the Office upon request and payment of the necessary fee.

FIG. 1 depicts a model for CRSBP-1 regulation in VE-cadherin-mediated intercellular adhesion in lymphatic endothelial cells (LECs). At the cell surface, disulfide-linked homodimeric CRSBP-1 forms complexes with monomeric PDGFβR and β-catenin. Upon CRSBP-1 ligand binding, CRSBP-1 is induced to form oligomers or have a conformational change, resulting in the formation of dimers or oligomers of PDGFβR. The PDGFβR becomes activated and undergoes tyrosine autophosphorylation. Activation of PDGFβR or activated PDGFβR-mediated signaling cascades lead to increased tyrosine phosphorylation of VE-cadherin at Tyr658 and Tyr731 and β-catenin at Tyr142, resulting in dissociation of β-catenin and p120-catenin from VE-cadherin (which transdimerizes between cells) and subsequent internalization of VE-cadherin. α-Catenin becomes dissociated from β-catenin, leading to uncoupling of VE-cadherin junctions from actin cytoskeleton, disruption of VE-cadherin-mediated intercellular adhesion and opening of intercellular junctions in LECs and increased interstitial-lymphatic transit in intact mice. Under the conditions of CRSBP-1 down regulation or null mutation, VE-cadherin-mediated intercellular adhesion (VE-cadherin transdimerization) is compromised.

FIG. 2 graphically illustrates that VEGF peptide and PDGF peptide enhance immunity of pseudorabies virus vaccine (PRV/Marker Gold). Pigs were vaccinated at day 0 and challenged with virulent wild-type virus at day 28 (week 4) post vaccination as marked by an arrow. Pigs which were not vaccinated were used as control. The titer of 512 (an average number from 6 pigs) is the maximal limit of the assay and the exact titer should be higher.

### SUMMARY OF THE INVENTION

One aspect of the invention encompasses an adjuvant composition. The adjuvant composition comprises at least one peptide. The at least one peptide comprises one or more cell-surface retention sequence (CRS) motifs which contain a cluster of basic amino acid residues (Arg, Lys, His).

A further aspect of the invention provides a vaccine composition. The vaccine composition comprises an adjuvant composition. The adjuvant composition generally comprises at least one peptide. The at least one peptide comprises one or more cell-surface retention sequence motifs. The vaccine composition also comprises one or more antigens.

Yet another aspect of the invention provides a method of enhancing an immune response to an antigen in a subject. The method generally comprises administering to a subject a vaccine composition. The vaccine composition comprises an adjuvant composition. The adjuvant composition generally comprises at least one peptide. The at least one peptide comprises one or more CRS motifs. The vaccine composition also comprises one or more antigens.

Another aspect of the invention provides an isolated peptide. The peptide generally comprises one or more CRS motifs.

Other features and aspects of the invention are described in more detail herein.

### DETAILED DESCRIPTION

In accordance with the present invention, adjuvants capable of augmenting the immune response to an antigen in a vaccine composition have been developed. Advantageously, adjuvants of the invention act at a rate-limiting step during an immune response to an antigen in a vaccine. While not wishing to be bound by theory, it is believed that the adjuvants of the present invention function as adjuvants by enhancing entry of immune cells from the interstitial space into lymphatic vessels and lymph nodes.

The inventors discovered a mechanism of controlling the traffic of immune cells and fluid from tissues to lymphatic vessels during an immune response. In this mechanism, polypeptides comprising one or more CRS motifs induce disruption (unbuttoning) of VE-cadherin-mediated intercellular discontinuous adhesion (termed “button-like structure”) between lymphatic lining cells, allowing the antigen-loaded dendritic cells to enter the lymphatic vessels. Polypeptides comprising one or more CRS motifs induce disruption of VE-cadherin-mediated intercellular discontinuous adhesion by interaction of the one or more CRS motifs with CRSBP-1 at lymphatic intercellular junctions. For additional information, see FIG. 1, and e.g., Hou et al., 2011 Journal of Cell Science 124: 1231-1244, the disclosure of which is incorporated herein in its entirety.

The inventors further discovered that synthetic peptides comprising one or more CRS motifs also induce disruption of VE-cadherin-mediated intercellular discontinuous adhesion by interaction of the CRS motif with CRSBP-1 at lymphatic intercellular junctions (Hou et al., 2011 Journal of Cell Science 124: 1231-1244). Importantly, the inventors also showed that, like their parent polypeptides, the synthetic oligopeptides comprising the CRS motifs also act like a token to gain entry for immune cells to the lumen of lymphatic vessels and its network.

As such, the present invention provides adjuvant compositions, vaccine compositions, and methods of enhancing an immune response to an antigen based on the adjuvants of the invention. Various aspects of the invention are described in further detail in the following sections.

### I. Adjuvant Compositions

One aspect of the present invention provides adjuvant compositions. The adjuvant compositions of the invention comprise at least one isolated peptide, the peptide comprising one or more CRS motifs. As used herein, the phrase “isolated peptide” and “peptide” are used interchangeably to describe peptides of the invention. Thus, the phrase “a peptide of the invention” specifically refers to an isolated peptide. In addition to being isolated, peptides of the invention are also not naturally occurring peptides. For example, while a peptide of the invention may comprise one or more CRS motifs from a naturally occurring polypeptide comprising one or more CRS motifs as described below in Section I(a), the peptide itself is not a naturally occurring peptide. Other aspects of the invention are described in further detail below.

### (a) Isolated Peptides Comprising a CRS Motif

A peptide of the present invention comprises one or more CRS motifs. A CRS motif may be any amino acid sequence capable of interacting with CRSBP-1 and inducing disruption of VE-cadherin-mediated intercellular discontinuous adhesion at lymphatic intercellular junctions. CRS motifs are known in the art, and may be as described in Carmeliet et al., 1999 Nat. Med. 5:495-502, Joukov et al., 1997 EMBO J. 16:3898-3911, LaRochelle et al., 1991 Genes Dev 5:1191-1199, and Ostman et al., 1991 Cell Regul 2:503-512, the disclosures of which are incorporated herein in their entirety.

In general, a minimal CRS motif may comprise seven or eight contiguous amino acid residues, wherein at least four of the seven or eight amino acid residues may be basic amino acid residues. For instance, 4, 5, 6, 7, or 8 amino acid residues of the seven or eight contiguous amino acid residues may be basic amino acid residues. The basic amino acids may be positioned anywhere in the peptide including, but not limited to, the N-terminus and C-terminus. Non limiting examples of basic amino acids include arginine, lysine, and histidine.

In some embodiments, a CRS motif comprises seven contiguous amino acid residues, wherein at least four of the seven amino acid residues are arginine, lysine, or histidine. In a preferred embodiment, a CRS motif comprises seven contiguous amino acid residues, wherein four of the seven amino acid residues are arginine, lysine, or histidine. In another preferred embodiment, a CRS motif comprises seven contiguous amino acid residues, wherein five of the seven amino acid residues are arginine, lysine, or histidine. In each of the above embodiments, the basic amino acids may be positioned anywhere in the peptide including, but not limited to, the N-terminus and C-terminus.

In other embodiments, a CRS motif comprises eight contiguous amino acid residues, wherein at least four of the eight amino acid residues are arginine, lysine, or histidine. In a preferred embodiment, a CRS motif comprises eight contiguous amino acid residues, wherein four of the eight amino acid residues are arginine, lysine, or histidine. In another preferred embodiment, a CRS motif comprises eight contiguous amino acid residues, wherein five of the eight amino acid residues are arginine, lysine, or histidine. In each of the above embodiments, the basic amino acids may be positioned anywhere in the peptide including, but not limited to, the N-terminus and C-terminus.

A peptide of the present invention may comprise 1, 2, 3, 4, 5, 6, 7, 8, 9, 10 or more CRS motifs. In some embodiments, a peptide of the invention comprises 1, 2, 3, or 4 CRS motifs. In other embodiments, a peptide of the invention comprises 4, 5, 6, or 7 CRS motifs. In yet other embodiments, a peptide of the invention comprises 7, 8, 9, 10 or more CRS motifs.

When a peptide comprises more than one CRS motif, two or more of the CRS motifs may be overlapping. Alternatively, when a peptide comprises more than one CRS motif, two or more of the CRS motifs may be contiguous. Also, when a peptide comprises more than one CRS motif, two or more of the CRS motifs may be separated by one or more amino acid residues. A peptide of the invention may also comprise more than one CRS motif, wherein the CRS motifs may be a combination of overlapping CRS motifs, contiguous CRS motifs, or CRS motifs separated by one or more amino acid residues.

When two or more CRS motifs are separated by one or more amino acid residues, the two or more CRS motifs may be separated by about 1, 2, 3, 4, 5, 6, 7, 8, 9, or 10 amino acid residues, by about 1, 2, 3, 4, or 5 amino acid residues, or by about 5, 6, 7, 8, 9, or 10 amino acid residues.

In some embodiments, a peptide comprises more than one CRS motif, and two or more of the CRS motifs are overlapping. In other embodiments, a peptide comprises more than one CRS motif, and two or more of the CRS motifs are contiguous. In yet other embodiments, a peptide comprises more than one CRS motif, and two or more of the CRS motifs are separated by one or more amino acid residues. In other embodiments, a peptide comprises more than one CRS motif, and the CRS motifs may be a combination of overlapping CRS motifs, contiguous CRS motifs, or CRS motifs separated by one or more amino acid residues.

A peptide of the invention may consist of one or more CRS motifs. Alternatively, a peptide of the invention may also comprise one or more amino acid residues in addition to one or more CRS motifs. Additional residues may be at either terminus of a peptide. In some embodiments, a peptide of the invention consists of one or more CRS motifs. In other embodiments, a peptide of the invention comprises one or more amino acid residues in addition to one or more CRS motifs. For instance, a peptide of the invention may comprise about 1, 2, 3, 4, 5, 6, 7, 8, 9, or about 10 amino acid residues in addition to one or more CRS motifs, preferably about 1, 2, 3, 4, 5, or about 6 amino acid residues in addition to one or more CRS motifs, more preferably 1, 2, or about 3 amino acid residues in addition to one or more CRS motifs. In a preferred embodiment, a peptide of the invention comprises one amino acid residue in addition to one or more CRS motifs. In a particularly preferred alternative of the embodiment, a peptide of the invention comprises one amino acid residue at the N terminus of the peptide in addition to one or more CRS motifs.

As will be recognized by those of skill in the art, a peptide of the invention may be of any length, provided that the peptide comprises one or more CRS motifs, is capable of augmenting the immune response to an antigen, and is capable of being used in an adjuvant composition for augmenting the immune response to an antigen. A peptide of the invention may be about 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or about 100, or more amino acids long. In some embodiments, a peptide of the invention is about 7, 8, 9, 10, 11, 12, 13, 14, or about 15 amino acids long. In other embodiments, a peptide of the invention is about 12, 13, 14, 15, 16, 17, 18, 19, or about 20 amino acids long. In yet other embodiments, a peptide of the invention is about 17, 18, 19, 20, 21, 22, 23, 24, or about 25 amino acids long. In still other embodiments, a peptide of the invention is about 22, 23, 24, 25, 26, 27, 28, 29, 30, 35, 40, 45, or about 50 amino acids long. In other embodiments, a peptide of the invention is about 40, 45, 50, 55, 60, 65, 70, 75, 80, 85, 90, 95, or about 100, or more amino acids long. In some preferred embodiments, a peptide of the invention is about 17, 18, 19, 20, or about 21, or more amino acids long. In other preferred embodiments, a peptide of the invention is about 23, 24, 25, 26, or about 27, or more amino acids long.

An adjuvant peptide of the invention comprising one or more CRS motifs may comprise one or more CRS motifs from a naturally occurring polypeptide comprising one or more CRS motifs. Alternatively, an adjuvant peptide may be a peptide comprising synthetic CRS motifs not normally present in a naturally occurring polypeptide.

In some embodiments, an adjuvant peptide is a peptide comprising synthetic CRS motifs not normally present in a naturally occurring polypeptide. A peptide comprising synthetic CRS motifs may be identified using its ability to specifically bind CRSBP-1. Such a peptide may be identified using biopanning of peptide libraries for isolation of peptides that specifically bind CRSBP-1. Methods of biopanning ligand libraries for isolation of peptides that specifically bind to a protein are known in the art, and may be as described in U.S. Pat. No. 7,968,675; U.S. Pat. No. 7,402,392; U.S. Pat. No. 7,906,102; U.S. Pat. No. 7,306,925; Jaboin et al., Methods in Molecular Biology, 542:285-300; Wang et al., PLoS One. 2010; 5(8): e12051; and International Publication WO2005042780, the disclosures of which are incorporated herein by reference in their entireties.

In other embodiments, an adjuvant peptide of the invention comprising one or more CRS motifs comprises one or more CRS motifs from a naturally occurring polypeptide. A number of polypeptides comprise one or more CRS motifs. For instance, a number of growth factors or cytokines that are capable of interacting with CRSBP-1 and are capable of cell-surface retention comprise one or more CRS motifs. Non limiting examples of naturally occurring polypeptides comprising one or more CRS motifs suitable for the invention may include platelet-derived growth factor B proto-oncogene (PDGF-B c-sis), platelet-derived growth factor B viral oncogene (PDGF-B v-sis), platelet-derived growth factor A_{L} (PDGF-A_{L}), vascular endothelial cell growth factor A (VEGF-A), platelet-derived growth factor D (PDGF-D), placenta growth factor (PIGF), insulin-like growth factor binding protein 3 (IGFBP-3), fibroblast growth factor 3 (FGF-3), proto-oncogene protein Wnt-1 (Wnt-1), cysteine-rich angiogenic inducer 61 (CYR 61), chemokine (C—C motif) ligand 21 (CCL 21), and interferon γ (IFN-γ).

In some embodiments, a peptide of the invention comprises one or more CRS motifs from placenta growth factor (PIGF). In other embodiments, a peptide of the invention comprises one or more CRS motifs from insulin-like growth factor binding protein 3 (IGFBP-3). In yet other embodiments, a peptide of the invention comprises one or more CRS motifs from fibroblast growth factor 3 (FGF-3). In additional embodiments, a peptide of the invention comprises one or more CRS motifs from proto-oncogene protein Wnt-1 (Wnt-1). In still other embodiments, a peptide of the invention comprises one or more CRS motifs from cysteine-rich angiogenic inducer 61 (CYR 61). In some embodiments, a peptide of the invention comprises one or more CRS motifs from chemokine (C—C motif) ligand 21 (CCL 21). In other embodiments, a peptide of the invention comprises one or more CRS motifs from interferon γ (IFN-γ). In some preferred embodiments, a peptide of the invention comprises one or more CRS motifs from platelet-derived growth factor A_{L} (PDGF-A_{L}). In other preferred embodiments, a peptide of the invention comprises one or more CRS motifs from platelet-derived growth factor D (PDGF-D). In particularly preferred embodiments, a peptide of the invention comprises one or more CRS motifs from platelet-derived growth factor B proto-oncogene (PDGF-B c-sis). In other particularly preferred embodiments, a peptide of the invention comprises one or more CRS motifs from platelet-derived growth factor B viral oncogene (PDGF-B v-sis). In yet other particularly preferred embodiments, a peptide of the invention comprises one or more CRS motifs from vascular endothelial cell growth factor A (VEGF-A).

In one embodiment, a peptide of the invention comprises RRRPKGRGKRRR (SEQ ID NO. 1). In another embodiment, a peptide of the invention comprises KKGFYKKKQCRPSKGRKR (SEQ ID NO. 2). In yet another embodiment, a peptide of the invention comprises KGRPRRGFKTRR (SEQ ID NO. 3). In another embodiment, a peptide of the invention comprises RKQRRLTR (SEQ ID NO. 4). In one embodiment, a peptide of the invention comprises KKGKKCSKTKK (SEQ ID NO. 5). In an additional embodiment, a peptide of the invention comprises KTGKKGKGSK (SEQ ID NO. 6). In another embodiment, a peptide of the invention comprises KTGKRKR (SEQ ID NO. 7). In a preferred embodiment, a peptide of the invention comprises KKRKRKRLK (SEQ ID NO. 8). In another preferred embodiment, a peptide of the invention comprises KRRGRAK (SEQ ID NO. 9). In a particularly preferred embodiment, a peptide of the invention comprises RTVRVRRPPKGKHRKFK (SEQ ID NO. 10). In a particularly preferred embodiment, a peptide of the invention comprises RTVRVRRPPKCKHRKFK (SEQ ID NO. 11).

In an exemplary embodiment, a peptide of the invention is YVRVRRPPKGKHRKFKHTH (SEQ ID NO. 12). In another exemplary embodiment, a peptide of the invention is KKSVRGKGKGQKRKRKKSRYKSWSV (SEQ ID NO. 13)

In addition to the peptide sequences shown in SEQ ID NO: 1-13, it will be appreciated by those skilled in the art that amino acid sequence polymorphisms may exist within a population (e.g., the human population). Such genetic polymorphism in CRS motifs may exist among individuals within a population due to natural allelic variation. Such natural allelic variations may result in as much as 15% variance in the amino acid sequence of a peptide of the invention comprising one or more CRS motifs. Any and all such amino acid variations and resulting polymorphisms in CRS motifs that are the result of natural allelic variation and that do not alter the functional activity of CRS motifs are intended to be within the scope of the invention. Thus, e.g., 1%, 2%, 3%, 4%, or 5% of the amino acids in CRS motifs may be replaced by another amino acid.

Moreover, CRS motifs from different species (CRS motif orthologs/homologues), which have an amino acid sequence which differs from that of CRS motifs disclosed herein, are intended to be within the scope of the invention.

In addition to naturally occurring allelic variants of CRS motifs that may exist in the population, the skilled artisan will further appreciate that changes may be introduced by mutation into the amino acid sequence of SEQ ID NO: 1-13, without altering the functional ability of the peptide.

Accordingly, another aspect of the invention pertains to peptides that contain changes in amino acids of CRS motifs that may or may not be essential for activity. Such peptide sequences differ from SEQ ID NO: 1-13. In one embodiment, a peptide includes an amino acid sequence that is at least about 45% identical, 65%, 75%, 85%, 90%, 91%, 92%, 93%, 94%, 95%, 96%, 97%, 98%, or more identical to the sequence of SEQ ID NO: 1-13.

A peptide of the invention may be subject to various changes, substitutions, insertions, and deletions where such changes provide for certain advantages in its use. Thus, the invention encompasses any of a variety of forms of peptide derivatives that include amides, conjugates with proteins, cyclized peptides, polymerized peptides, conservatively substituted variants, analogs, fragments, peptoids, chemically modified peptides, and peptide mimetics.

Peptides of the invention may comprise naturally occurring amino acids, synthetic amino acids, genetically encoded amino acids, non-genetically encoded amino acids, and combinations thereof. Peptides may include both L-form and D-form amino acids.

Representative non-genetically encoded amino acids may include but are not limited to: 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids may include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters, or other types of esters or hydrazides. Free hydroxyl groups can be derivatized to form O-acyl or O-alkyl derivatives. The imidazole nitrogen of histidine can be derivatized to form N-im-benzylhistidine.

The peptides may further comprise conservatively substituted variants of the peptides described above. The term “conservatively substituted variant” may refer to a peptide wherein one or more residues have been conservatively substituted with a functionally similar residue and which displays the adjuvant activity as described herein. The phrase “conservatively substituted variant” also includes peptides wherein a residue is replaced with a chemically derivatized residue, provided that the resulting peptide displays adjuvant activity as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Peptides of the present invention also include peptides comprising one or more additions and/or deletions or residues relative to the sequence of a peptide whose sequence is disclosed herein, so long as the requisite targeting activity of the peptide is maintained. The term “fragment” refers to a peptide comprising an amino acid residue sequence shorter than that of a peptide disclosed herein.

Additional residues may also be added at either terminus of a peptide for the purpose of providing a “linker” by which the peptides of the present invention can be conveniently affixed to a label or solid matrix, or carrier. Amino acid residue linkers are usually at least one residue and may be 40 or more residues, more often 1 to 10 residues. Typical amino acid residues used for linking are tyrosine, cysteine, lysine, glutamic and aspartic acid, or the like. In addition, a peptide may be modified by terminal-NH2 acylation (e.g., acetylation, or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong half-life of the peptides in solutions, particularly biological fluids where proteases may be present.

Peptide cyclization is also a useful terminal modification, and is particularly preferred also because of the stable structures formed by cyclization and in view of the biological activities observed for such cyclic peptides as described herein. An exemplary method for cyclizing peptides is described by Schneider & Eberle (1993) Peptides, 1992: Proceedings of the Twenty-Second European Peptide Symposium, Sep. 13-19, 1992, Interlaken, Switzerland, Escom, Leiden. Typically, tertbutoxycarbonyl protected peptide methyl ester is dissolved in methanol and sodium hydroxide solutions are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxyl termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

The term “peptoid” as used herein refers to a peptide wherein one or more of the peptide bonds are replaced by pseudopeptide bonds including, but not limited to, a carba bond (CH2—CH2), a depsi bond (CO—O), a hydroxyethylene bond (CHOH—CH2), a ketomethylene bond (CO—CH2), a methylene-oxy bond (CH2—O), a reduced bond (CH2—NH), a thiomethylene bond (CH2—S), a thiopeptide bond (CS—NH), and an N-modified bond (—NRCO—). See e.g., Corringer et al. (1993) J Med Chem 36:166-172; Garbay-Jauregiuberry et al. (1992) Int J Pept Protein Res 39:523-527; Tung et al. (1992) Pept Res 5:115-118; Urge et al. (1992) Carbohydr Res 235:83-93; Pavone et al. (1993) Int J Pept Protein Res 41:15-20.

Peptides of the present invention, including peptoids, may be synthesized by any of the techniques that are known to those skilled in the art of peptide synthesis. Synthetic chemistry techniques, such as a solid-phase Merrifield-type synthesis, may be preferred for reasons of purity, antigenic specificity, freedom from undesired side products, ease of production and the like. A summary of representative techniques can be found in Stewart & Young (1969) Solid Phase Peptide Synthesis, Freeman, San Francisco; Merrifield (1969) Adv Enzymol Relat Areas Mol Biol 32:221-296; Fields & Noble (1990) Int J Pept Protein Res 35:161-214; and Bodanszky (1993) Principles of Peptide Synthesis. 2nd rev. ed. Springer-Verlag, Berlin, New York. Solid phase synthesis techniques can be found in Andersson et al. (2000) Biopolymers 55:227-250, references cited therein, and in U.S. Pat. Nos. 6,015,561, 6,015,881, 6,031,071, and 4,244,946. Peptide synthesis in solution is described by Schroder & Lübke (1965) The Peptides, Academic Press, New York. Appropriate protective groups usable in such synthesis are described in the above texts and in McOmie (1973) Protective Groups in Organic Chemistry, Plenum Press, London, New York. Peptides that include naturally occurring amino acids can also be produced using recombinant DNA technology. In addition, peptides comprising a specified amino acid sequence can be purchased from commercial sources (e.g., Biopeptide Co., LLC of San Diego, Calif. and PeptidoGenics of Livermore, Calif.).

Any peptide or peptide mimetic of the present invention may be used in the form of a pharmaceutically acceptable salt. Suitable acids which are capable of forming a pharmaceutically acceptable salt with the peptides of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Suitable bases capable of forming salts with the peptides of the present invention include inorganic bases such as sodium hydroxide, ammonium hydroxide, potassium hydroxide and the like; and organic bases such as mono-di- and tri-alkyl and aryl amines (e.g., triethylamine, diisopropyl amine, methyl amine, dimethyl amine and the like), and optionally substituted ethanolamines (e.g., ethanolamine, diethanolamine and the like).

### (b) Compositions

In general, peptides of the invention are used as adjuvants for augmenting the immune response when used in combination with an antigen in a vaccine composition. As such, peptides of the invention may be formulated in an adjuvant composition suitable for use in a vaccine. Alternatively, a peptide of the invention may be directly formulated with the vaccine to form a vaccine formulation comprising a peptide of the invention. Vaccine formulations may be as described in Section II below.

An adjuvant composition of the invention may comprise one or more than one peptide of the invention. For instance, an adjuvant composition of the invention may comprise about 1, about 2, about 3, about 4, about 5, about 6, about 7, about 8, about 9, or about 10 or more peptides of the invention. In some embodiments, an adjuvant composition of the invention comprises about 1, 2, 3, 4, or about 5 peptides of the invention. In other embodiments, an adjuvant composition of the invention comprises about 5, 6, 7, 8, 9, or about 10 peptides of the invention. In a preferred embodiment, an adjuvant composition of the invention comprises one peptide of the invention.

The amount of the peptide of the invention that may be combined with materials to produce a single dose of an adjuvant composition can and will vary depending upon the subject, the peptide, the formulation, and the particular mode of administration. Those skilled in the art will appreciate that dosages may also be determined with guidance from Goodman & Goldman's The Pharmacological Basis of Therapeutics, Ninth Edition (1996), Appendix II, pp. 1707-1711, and from Goodman & Goldman's The Pharmacological Basis of Therapeutics, Tenth Edition (2001), Appendix II, pp. 475-493.

In general, the amount of peptide in a single dose of an adjuvant composition may be about 1, 10, 20, 30, 40, 50, 60, 70, 80, 90, 100, 110, 120, 130, 140, 150, 160, 170, 180, 190, or about 200 fold or more excess peptide sufficient to inhibit binding of ¹²⁵I-labeled peptide to CRSBP-1 expressed in H1299 cells. In some embodiments, the amount of peptide in a single dose of an adjuvant composition is about 1, 10, 20, 30, 40, or about 50, about 40, 50, 60, 70, 80, 90, or about 100, about 80, 90, 100, 110, 120, 130, 140, or about 150, or about 150, 160, 170, 180, 190, or about 200 fold or more excess peptide sufficient to inhibit binding of ¹²⁵I-labeled peptide to CRSBP-1 expressed in H1299 cells. In preferred embodiments, the amount of peptide in a single dose of an adjuvant composition is about 80, 90, 100, 110, 120, 130, 140, or about 150 fold or more excess peptide sufficient to inhibit binding of ¹²⁵I-labeled peptide to CRSBP-1 expressed in H1299 cells. Determining the amount of peptide sufficient to inhibit binding of ¹²⁵I-labeled peptide to CRSBP-1 expressed in H1299 cells may be as described in Huang et al., 2003 J Biol Chem 278:43855-43869, the disclosure of which is incorporated herein in its entirety.

### II. Vaccine Compositions

In other aspects, the present invention encompasses a vaccine composition. A vaccine composition of the invention comprises an adjuvant composition of the invention and one or more antigens. A wide variety of antigens may find use in conjunction with the compositions and formulations of the present invention. In particular, adjuvant compositions provided herein may be advantageously combined with antigenic stimulation from tumor-associated antigens or pathogen antigens to enhance an immune response against the corresponding tumor or pathogen. Generally, suitable antigens may be derived from proteins, peptides, polypeptides, lipids, glycolipids, carbohydrates, and DNA found in the subject tumor or pathogen. Such antigenic stimulation may come from tumor-associated antigens, pathogen antigens, and autoantigens.

In some embodiments, an antigen of the invention is a tumor-associated antigen. In other embodiments, an antigen of the invention is a pathogen antigen. An adjuvant composition may be as described in Section I. Antigens and vaccine compositions may be as described herein below.

### (a) Tumor Associated Antigen

An antigen of the invention may be a tumor-associated antigen. Tumor-associated antigens finding utility herein may include both mutated and non-mutated molecules which may be indicative of a single tumor type, shared among several types of tumors, and/or exclusively expressed or over-expressed in tumor cells in comparison with normal cells. In addition to proteins and glycoproteins, tumor-specific patterns of expression of carbohydrates, gangliosides, glycolipids and mucins have also been documented.

Exemplary tumor-associated antigens for use in the subject cancer vaccines include protein products of oncogenes, tumor suppressor genes and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins, and a number of other self-proteins.

For instance, tumor-associated antigens may include, e.g., mutated antigens such as the protein products of the Ras p21 protooncogenes, tumor suppressor p53 and HER-2/neu and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self-antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas.

Tumor-associated antigens and their respective tumor cell targets may include, e.g., cytokeratins, particularly cytokeratin 8, 18 and 19, as antigens for carcinoma. Epithelial membrane antigen (EMA), human embryonic antigen (HEA-125), human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens may be encoded by human papilloma virus. Chromagranin-A and synaptophysin are antigens of neuroendocrine and neuroectodermal tumors. Of particular interest are aggressive tumors that form solid tumor masses having necrotic areas. The lysis of such necrotic cells is a rich source of antigens for antigen-presenting cells, and thus the subject compositions and methods may find advantageous use in conjunction with conventional chemotherapy and/or radiation therapy.

Tumor-associated antigens may be prepared by methods well known in the art. For example, these antigens may be prepared from cancer cells either by preparing crude extracts of cancer cells (e.g., as described in Cohen et al., Cancer Res., 54:1055 (1994)), by partially purifying the antigens, by recombinant technology, or by de novo synthesis of known antigens. The antigen may also be in the form of a nucleic acid encoding an antigenic peptide in a form suitable for expression in a subject and presentation to the immune system of the immunized subject. Further, the antigen may be a complete antigen, or it may be a fragment of a complete antigen comprising at least one epitope.

Antigens derived from pathogens known to predispose to certain cancers may also be advantageously utilized in conjunction with the compositions and methods provided herein. It is estimated that close to 16% of the worldwide incidence of cancer can be attributed to infectious pathogens, and a number of common malignancies are characterized by the expression of specific viral gene products. Thus, the inclusion of one or more antigens from pathogens implicated in causing cancer may help broaden the host immune response and enhance the prophylactic or therapeutic effect of the cancer vaccine. Pathogens of particular interest for use herein include the hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLV1 (adult T cell leukemia), oncogenic human papilloma viruses types 16, 18, 33, 45 (adult cervical cancer), and the bacterium Helicobacter pylori (B cell gastric lymphoma).

### (b) Tumor

Tumor associated antigens that may be used in a vaccine composition of the invention may be any antigen that may treat or recognize a tumor derived from a neoplasm or a cancer. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

### (c) Pathogen Antigen

An antigen of the invention may be a pathogen antigen. Pathogen antigens may be derived from infectious microbes such as virus, bacteria, parasites and fungi and fragments thereof, in order to increase lymphocyte activity in response to active infection or improve the efficacy of prophylactic vaccine therapy. Examples of infectious virus include, but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, pseudorabies virus, cytomegalovirus (CMV), herpes virus; Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Also, gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of pathogens also include, but are not limited to, infectious fungi that infect mammals, and more particularly humans. Examples of infectious fungi include, but are not limited to: Cryptococcus neoformans, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e., protists) include Toxoplasma gondii.

Other medically relevant microorganisms that serve as antigens in mammals, and more particularly humans, are described extensively in the literature, e.g., see C. G. A Thomas, Medical Microbiology, Bailliere Tindall, Great Britain 1983, the entire contents of which is hereby incorporated by reference. In addition to the treatment of infectious human diseases, the compositions and methods of the present invention are useful for treating infections of non-human mammals. Many vaccines for the treatment of non-human mammals are disclosed in Bennett, K. Compendium of Veterinary Products, 3rd ed. North American Compendiums, Inc., 1995.

### (d) Composition

A vaccine composition comprising an adjuvant composition of the invention may optionally comprise one or more possible additives, such as carriers, preservatives, stabilizers, adjuvants in addition to peptide adjuvants of the invention, and other substances.

In one embodiment, a vaccine composition of the invention further comprises an adjuvant in addition to peptide adjuvants of the invention. Adjuvants, such as aluminum hydroxide or aluminum phosphate, may be optionally added to further increase the ability of the vaccine to trigger, enhance, or prolong an immune response. The vaccine compositions may further comprise additional components known in the art to improve the immune response to a vaccine, such as T cell co-stimulatory molecules or antibodies, such as anti-CTLA4. Additional materials, such as cytokines, chemokines, and bacterial nucleic acid sequences naturally found in bacteria, like CpG, are also potential vaccine adjuvants. In a preferred embodiment, a vaccine composition of the invention further comprises alum adjuvant in addition to peptide adjuvants of the invention.

In another embodiment, the vaccine may comprise a pharmaceutical carrier (or excipient). Such a carrier may be any solvent or solid material for encapsulation that is non-toxic to the inoculated host and compatible with the recombinant bacterium. A carrier may give form or consistency, or act as a diluent. Suitable pharmaceutical carriers may include liquid carriers, such as normal saline and other non-toxic salts at or near physiological concentrations, and solid carriers not used for humans, such as talc or sucrose, or animal feed. Carriers may also include stabilizing agents, wetting and emulsifying agents, salts for varying osmolarity, encapsulating agents, buffers, and skin penetration enhancers. Carriers and excipients, as well as formulations for parenteral and nonparenteral drug delivery, are set forth in Remington's Pharmaceutical Sciences 19th Ed. Mack Publishing (1995). When used for administering via the bronchial tubes, the vaccine is preferably presented in the form of an aerosol.

The dosages of a vaccine composition of the invention can and will vary depending on the adjuvant composition, the regulated antigen, and the intended host, as will be appreciated by one of skill in the art. Generally speaking, the dosage need only be sufficient to elicit a protective immune response in a majority of hosts. Routine experimentation may readily establish the required dosage. Administering multiple dosages may also be used as needed to provide the desired level of protective immunity.

A vaccine composition of the invention may also be a commercially available vaccine composition, wherein the commercially available vaccine composition is supplemented with an adjuvant composition of the invention.

### III. Methods

In yet other aspects, the invention encompasses methods of enhancing an immune response to an antigen in a subject. A method of the invention comprises enhancing an immune response to an antigen by administering to a subject a vaccine composition of the invention. Immune responses to antigens are well studied and widely reported. A vaccine composition of the invention may be as described in Section II.

As used herein, the tem “subject” may refer to a living organism having an immune system. In particular, subjects may include, but are not limited to, human subjects or patients and companion animals. Exemplary companion animals may include domesticated mammals (e.g., dogs, cats, horses), mammals with significant commercial value (e.g., dairy cows, beef cattle, pigs, sporting animals), mammals with significant scientific value (e.g., captive or free specimens of endangered species), or mammals which otherwise have value. Suitable subjects may also include: mice, rats, dogs, cats, ungulates such as cattle, swine, sheep, horses, and goats, lagomorphs such as rabbits and hares, other rodents, and primates such as monkeys, chimps, and apes. In some preferred embodiments, a subject is a human. In other preferred embodiments, a subject is a pig. Subjects may be of any age, including newborn, adolescent, adult, middle age, or elderly.

A vaccine composition may produce an immune response to an antigen in the vaccine upon administration of the vaccine to a subject. Methods of measuring an immune response to an antigen are known in the art and may include determining the titer of antibodies that bind the antigen in the serum of a vaccinated subject.

In general, the titer of antibodies that bind an antigen in the serum of a subject may be determined after allowing sufficient time after vaccination for the immune response to respond to the vaccination, therefore changing the titer of the antibody in the serum. As will be recognized by those of skill in the art, serum titer may be determined about 1, 2, 3, 4, 5, 6, or about 7 days after vaccination, about 1, 2, 3, 4, 5, 6, 7, 8, 9, or about 10 weeks after vaccination, or about 1, 2, 3, 4, 5, 6, 7, 8, 9, 10, 11, or about 12 months or more after vaccination. In some embodiments, serum titer is determined about 1, 2, or about 3 weeks after vaccination. In other embodiments, serum titer is determined about 6, 7, 8, 9, or about 10 weeks after vaccination.

As such, administering to a subject a vaccine composition comprising an adjuvant composition of the invention may increase the serum titer of antibodies that bind an antigen in the vaccine by about 1, 2, 3, 4, 5, 6, 7, 8, 9, 10, 11, 12, 13, 14, 15, 16, 17, 18, 19, or about 20 fold or more, when compared to the serum titer when administering a vaccine composition that does not comprise an adjuvant composition of the invention. In some embodiments, administering to a subject a vaccine composition comprising an adjuvant composition of the invention increases the serum titer of antibodies that bind an antigen in the vaccine by about 1, 2, 3, 4, or about 5 fold, by about 4, 5, 6, 7, 8, 9, or about 10 fold, about 5 fold, by about 9, 10, 11, 12, 13, 14, or about 15 fold, by about 13, 14, 15, 16, 17, 18, 19, or about 20 fold or more, when compared to the serum titer when administering a vaccine composition that does not comprise an adjuvant composition of the invention. In one embodiment, administering to a subject a vaccine composition comprising an adjuvant composition of the invention increases the serum titer of antibodies that bind an antigen in the vaccine by about 1, 2, 3, 4, or about 5 fold about two weeks after vaccination, when compared to the serum titer when administering a vaccine composition that does not comprise an adjuvant composition of the invention. In another embodiment, administering to a subject a vaccine composition comprising an adjuvant composition of the invention increases the serum titer of antibodies that bind an antigen in the vaccine by about 1, 2, 3, 4, or about 5 fold about eight weeks after vaccination, when compared to the serum titer when administering a vaccine composition that does not comprise an adjuvant composition of the invention. In another embodiment, administering to a subject a vaccine composition comprising an adjuvant composition of the invention increases the serum titer of antibodies that bind an antigen in the vaccine by about 7, 8, 9, 10, 11, or about 12 fold about two weeks after vaccination, when compared to the serum titer when administering a vaccine composition that does not comprise an adjuvant composition of the invention.

Enhancing an immune response to an antigen in a subject may extend the survival of the subject. Alternatively, enhancing an immune response to an antigen in a subject may cure the subject from a cancer or pathogen for which a vaccine is formulated. Enhancing an immune response to an antigen in a subject may also improve the health of the subject. Additionally, enhancing an immune response to an antigen in a subject may prevent the subject from acquiring a cancer or pathogen for which a vaccine is formulated. Alternatively, enhancing an immune response to an antigen in a subject may limit the duration and/or severity of the disease in the subject resulting from cancer or pathogen for which a vaccine is formulated.

Vaccine compositions of the invention may be formulated into pharmaceutical compositions and administered by a number of different means that may deliver a therapeutically effective dose. Such compositions may be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In preferred embodiments, vaccine compositions of the invention are formulated for intramuscular administration. Formulation of vaccines is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Vaccine compositions suitable for injectable use may include sterile aqueous solutions (where water soluble) or dispersions and sterile powders for the extemporaneous preparation of sterile injectable solutions or dispersion. Suitable carriers include physiological saline, bacteriostatic water, Cremophor EL (BASF; Parsippany, N.J.), or phosphate buffered saline (PBS). In all cases, a composition may be sterile and may be fluid to the extent that easy syringeability exists. A composition may be stable under the conditions of manufacture and storage and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Solid dosage forms for oral administration may include capsules, tablets, pills, powders, and granules. In such solid dosage forms, the compound is ordinarily combined with one or more adjuvants appropriate to the indicated route of administration. If administered per os, the compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as can be provided in a dispersion of active compound in hydroxypropylmethyl cellulose. In the case of capsules, tablets, and pills, the dosage forms may also comprise buffering agents such as sodium citrate, or magnesium or calcium carbonate or bicarbonate. Tablets and pills may additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

### IV. Kits

In still other aspects, the present invention provides articles of manufacture and kits containing materials useful for treating the conditions described herein. The article of manufacture may include a container of a composition as described herein with a label. Suitable containers include, for example, bottles, vials, and test tubes. The containers may be formed from a variety of materials such as glass or plastic. The container holds a composition having an adjuvant composition of the invention which is effective for vaccination. The active agent is at least one peptide of the invention comprising one or more CRS motifs, and may further include additional bioactive agents known in the art for treating the specific condition. The label on the container may indicate that the composition is useful for treating specific conditions and may also indicate directions for administration.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, “administering” is used in its broadest sense to mean contacting a subject with a composition of the invention.

As used herein, a “pharmaceutical composition” includes a pharmacologically effective amount of a therapeutic agent of the invention and a pharmaceutically acceptable carrier. As used herein, “pharmacologically effective amount,” “therapeutically effective amount” or simply “effective amount” refers to that amount of an agent effective to produce the intended pharmacological, therapeutic or preventive result. For example, if a given clinical treatment is considered effective when there is at least a 15% reduction in a measurable parameter associated with a disease or disorder, a therapeutically effective amount of an agent for the treatment of that disorder or disease is the amount necessary to effect at least a 15% reduction in that parameter.

The term “pharmaceutically acceptable carrier” refers to a carrier for administration of a therapeutic agent. Such carriers may include, but are not limited to, saline, buffered saline, dextrose, water, glycerol, ethanol, and combinations thereof. The term specifically excludes cell culture medium. For drugs administered orally, pharmaceutically acceptable carriers may include, but are not limited to, pharmaceutically acceptable excipients such as inert diluents, disintegrating agents, binding agents, lubricating agents, sweetening agents, flavoring agents, coloring agents and preservatives. Suitable inert diluents may include sodium and calcium carbonate, sodium and calcium phosphate, and lactose, while corn starch and alginic acid are suitable disintegrating agents. Binding agents may include starch and gelatin, while the lubricating agent, if present, may generally be magnesium stearate, stearic acid or talc. If desired, the tablets may be coated with a material such as glyceryl monostearate or glyceryl distearate to delay absorption in the gastrointestinal tract.

In practicing the present invention, many conventional techniques in molecular biology, microbiology, and recombinant DNA may be used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins eds.); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian cells, 1987 (J. H. Miller and M. P. Calos eds., Cold Spring Harbor Laboratory); and Methods in Enzymology, Vol. 154 and Vol. 155 (Wu and Grossman, and Wu, eds., respectively).

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction for Example 1

Lymphatic entry of immune cells is known to be an important rate-limiting step in primary immunity to viruses or tumor cells. As such, promoting vaccine potency by enhancing migration or transit of immune cells (e.g., antigen-loaded dendritic cells at the vaccine injection site) from the interstitial space into lymphatic vessels and lymph nodes presents an attractive pathway for adjuvant action. However, none of all known adjuvants, including alum, acts at this rate-limiting step. The modes of action of known adjuvants include: 1) the formation of a depot of antigen at the site of inoculation, with slow release; 2) the presentation of antigen to immunocompetent cells; and 3) the stimulation of production of various and different lymphokines (interleukins and tumor necrosis factor).

The inventors recently identified a novel mechanism to control the traffic of immune cells and fluid from tissues to lymphatic vessels during an immune response (Hou et al., 2011 Journal of Cell Science 124: 1231-1244). In this mechanism, antigen-loaded dendritic cells enter the lymphatic vessels by releasing molecules that induce disruption of VE-cadherin-mediated intercellular discontinuous adhesion (termed “button”-like structure) between lymphatic lining cells, allowing the antigen-loaded dendritic cells to enter the lymphatic vessels. These molecules are a group of growth factors (e.g., VEGF-A and PDGF-BB) and cytokines (e.g., IGFBP-3, CCL21 and IFN-γ). All growth factors and cytokines that are capable of inducing disruption of VE-cadherin-mediated intercellular discontinuous adhesion, possess cell-surface retention sequence (CRS) motifs. CRS motifs comprise specific clustered basic amino acid residues (Arg, Lys and His) and are evolutionarily conserved. The CRS-containing growth factors/cytokines are putative physiological ligands of CRS binding protein-1 (CRSBP-1), which is a membrane glycoprotein receptor and is localized at the cell surface of lymphatic endothelial cells (LECs). During an immune response, these growth factors and cytokines are secreted by immune cells, and interact with CRSBP-1 at lymphatic intercellular junctions. The interaction of the CRS-containing growth factors and cytokines with CRSBP-1 at lymphatic intercellular junctions acts like a token to gain entry for the immune cells to the lumen of lymphatic vessels and its network, by inducing opening of lymphatic intercellular junctions (FIG. 1; Hou et al., 2011 Journal of Cell Science 124: 1231-1244).

The inventors previously showed that synthetic oligopeptides containing the CRS motifs of growth factors/cytokines also serve as specific ligands of CRSBP-1 (Hou et al., 2011 Journal of Cell Science 124: 1231-1244). Importantly, the inventors also showed that, like their parent polypeptides, the synthetic oligopeptides containing the CRS motifs can also act like a token to gain entry for the immune cells to the lumen of lymphatic vessels and its network. For example, a synthetic 25-mer VEGF peptide (KKSVRGKGKGQKRKRKKSRYKSWSV; SEQ ID NO: 13), and a synthetic 19-mer PDGF peptide (YVRVRRPPKGKHRKFKHTH; SEQ ID NO: 12) containing the CRS motifs of VEGF-A and PDGF-B, respectively, bind to CRSBP-1 with high affinity in LECs (SVEC4-10 cells) as determined by ¹²⁵I-labeled VEGF peptide and PDGF peptide affinity labeling (Hou et al., 2011 Journal of Cell Science 124: 1231-1244; Boensch et al., 1995 J. Biol. Chem. 270:1807-1816). In fact, all synthetic peptides shown in Table 1 comprise one or more CRS motifs, and at 100-fold excess, inhibit binding of ¹²⁵I-labeled VEGF peptide and PDGF peptide to CRSBP-1 expressed in H1299 cells (Huang et al., 2003 J Biol Chem 278:43855-43869), suggesting that all these peptides can serve as specific ligands of CRSBP-1. Importantly, these peptides do not bind to their respective parent growth factor or cytokine receptors.

TABLE 1
Putative CRSs of secretary growth
factors and cytokines.

| | CRS. Numbers indicate amino |
|---|---|
| Polypeptide | acid number in the polypeptide |
| PDGF-B | |
| c-sis | 211RTVRVRRPPKGKHRKFK227 |
| | (SEQ ID NO: 10) |
| v-sis | 241RTVRVRRPPKCKHRKFK257 |
| | (SEQ ID NO: 11) |
| PDGF-A_{L} | 201KKRKRKRLK209 |
| | (SEQ ID NO: 8) |
| VEGF-A | 115KKSVRGKGKGQKRKRKKSR135 |
| | (SEQ ID NO: 14) |
| PDGF-D | 339KRRGRAK345 |
| | (SEQ ID NO: 9) |
| PIGF | 141RRRPKGRGKRRR152 |
| | (SEQ ID NO: 1) |
| FGF-3 (Int-2) | 163KGRPRRGFKTRR174 |
| | (SEQ ID NO: 3) |
| | 209RQRRQKK215 |
| | (SEQ ID NO: 15) |
| IGFBP-3 | 242KKGFYKKKQCRPSKGRKR259 |
| | (SEQ ID NO: 2) |
| Wnt-1 | 66RKQRRLTR73 |
| | (SEQ ID NO: 4) |
| Cyr 61 | 279KKGKKCSKTKK289 |
| | (SEQ ID NO: 5) |
| CCL21 | 111KTGKKGKGSK120 |
| | (SEQ ID NO: 6) |
| IFN-γ | 168KTGKRKR174 |
| | (SEQ ID NO: 7) |

PDGF, platelet-derived growth factor; c-sis, proto-oncogene of sis; v-sis, viral oncogene
of sis; VEGF, vascular endothelial cell growth factor; PIGF, placenta growth factor;
IGFBP, insulin-like growth factor binding protein; FGF, fibroblast growth factor; IFN,
interferon

In Hou et al. (2011 Journal of Cell Science 124: 1231-1244), the inventors also demonstrated that, like VEGF-A and PDGF-BB, VEGF peptide and PDGF peptide induce disruption of VE-cadherin-mediated discontinuous intercellular adhesion and opening of intercellular junctions in lymphatic endothelial cell (LEC) monolayers as determined by immunofluorescence microscopy and Transwell permeability assay. This occurs by interaction of the peptides with CRSBP-1 in the CRSBP-1-PDGFβR (PDGF β-type receptor)-β-catenin complex, resulting in tyrosine phosphorylation of the complex, dissociation of β-catenin and p120-catenin from VE-cadherin, and internalization of VE-cadherin (FIG. 1; Hou et al., 2011 Journal of Cell Science 124: 1231-1244). Pretreatment of LECs with a PDGFβR kinase inhibitor abolishes ligand-stimulated tyrosine phosphorylation of VE-cadherin, halts the ligand-induced disruption of VE-cadherin intercellular adhesion, and blocks the ligand-induced opening of intercellular junctions (Hou et al., 2011 Journal of Cell Science 124: 1231-1244, Hou et al., 2012 FEBS Lett 586:1480-1487). These CRSBP-1 ligands also induce opening of lymphatic intercellular junctions that respond to PDGFβR kinase inhibitor in wild-type mice as evidenced by increased transit of injected FITC-dextran and induced edema fluid from the interstitial space into lymphatic vessels (Hou et al., 2011 Journal of Cell Science 124: 1231-1244). In the control experiments, polyarginine and polylysine exhibit very weak activity as compared to VEGF peptide and PDGF peptide.

The Examples presented herein below, show that peptides comprising CRS motifs function as adjuvants by enhancing lymphatic entry of immune cells at the vaccine injection site

### Example 1

### Peptides Comprising CRS Motifs Potentiate Efficacy of Commercially Available Vaccines

It was hypothesized that the adjuvants (PDGF peptide and VEGF peptide) may potentiate efficacy of commercially available vaccines, even though these commercially available vaccines may contain alum as an adjuvant. This is because the peptide adjuvants of the present invention and alum augment immune responses by distinct mechanisms. To test this hypothesis, the effects of the peptide adjuvants were determined on the efficacy of a commercially available pseudorabies virus (PRV) vaccine (PRV/Marker Gold*; manufacturer: Intervet/Schering-Plough Animal Health) for pigs. PRV/Marker Gold* vaccine is a genetically designed, live virus vaccine for the immunization of healthy pigs for prevention of disease caused by pseudorabies virus (Aujeszky's disease).

Twenty-four pigs (10 weeks old) were divided into 4 groups (6 pigs per group). Pigs were vaccinated intramuscularly with 1 ml of PRV/Marker Gold* alone, PRV/Marker Gold* alone ±200 μg PDGF peptide (PDGF) or VEGF peptide (VEGF) or normal saline (vaccine alone). All pigs in each group were challenged with a virulent strain (termed TNL) of PRV virus isolate at day 28 (week 4) post vaccination. The challenge dosage was 10⁵ TCID₅₀ in a 2 ml volume. The titers of the antibody to PSV virus in the sera of pigs were estimated using standard procedures according to the protocol of the manufacturer.

As shown in FIG. 2, pigs vaccinated with the vaccine containing VEGF peptide and PDGF peptide exhibited greatly increased serum antibody titer at day 14 (week 2) post vaccination (˜9 fold and ˜4 fold, respectively, as compared to those vaccinated with vaccine only), and two weeks after challenge with wild-type virulent virus (>3 fold for both VEGF peptide and PDGF peptide as compared to those vaccinated with vaccine only). It is important to note that both PDGF peptide and VEGF peptide potentiate immunity (>3 fold) of PRV vaccine even after challenge with virulent PRV. This suggests that the effects of these peptides are still in the memory of immune cells at least for 8 weeks after vaccination. No known adjuvants have been reported to exhibit such novel activity.

PDGF peptide and VEGF peptide also protected the animals from mortality caused by challenge with virulent PRV. The survival rates of pigs were determined at day 56 (week 8). All control animals died one week after challenge with virulent wild-type virus (week 5), but none of the animals died when vaccinated using a peptide adjuvant of the invention (Table 2).

TABLE 2
Mortality of pigs immunized with pseudorabies virus vaccine
with and without VEGF peptide and PDGF peptide 4 weeks
after challenge with virulent wild-type virus.

| Vaccine immunization* | Survival (number of pigs) |
|---|---|
| No vaccine | 0 |
| Vaccine + vehicle | 5* (*one pig died 1 week after challenge) |
| Vaccine + VEGF peptide | 6 |
| Vaccine + PDGF peptide | 6 |

In conclusion, oligopeptides comprising a CRS motif enhance efficacy of vaccines by enhancing primary immunity at an important rate-limiting step that is distinct from all known adjuvants to date. In addition, oligopeptides adjuvants described herein are safe.

What is claimed is:

1. An adjuvant composition comprising at least one isolated peptide, the peptide comprising one or more cell-surface retention sequence (CRS) motifs.

2. The composition of claim 1, wherein the peptide comprises one CRS motif.

3. The composition of claim 1, wherein the number of CRS motifs in a peptide is selected from the group consisting of 2, 3, 4, and 5 CRS motifs.

4. The composition of claim 3, wherein the CRS motifs are overlapping or contiguous.

5. The composition of claim 1, wherein the CRS motif is selected from the group consisting of (i) a CRS motif comprising seven contiguous amino acid residues, wherein at least four of the seven amino acid residues are arginine, lysine, or histidine; (ii) a CRS motif comprising seven contiguous amino acid residues, wherein at least five of the eight amino acid residues are arginine, lysine, or histidine; (iii) a CRS motif comprising eight contiguous amino acid residues, wherein at least four of the seven amino acid residues are arginine, lysine, or histidine; and (iv) a CRS motif comprising eight contiguous amino acid residues, wherein at least five of the eight amino acid residues are arginine, lysine, or histidine.

6. The composition of claim 1, wherein the peptide comprises a sequence selected from the group consisting of SEQ ID NO: 1 to SEQ ID NO: 15, inclusive.

7. The composition of claim 1, wherein the peptide consists of a sequence selected from the group consisting of SEQ ID NO: 1 to SEQ ID NO: 15, inclusive.

8. The composition of claim 1, wherein the peptide is selected from the group consisting of YVRVRRPPKGKHRKFKHTH (SEQ ID NO: 12) and KKVRGKGKGQKRKRKKSRYKSWSV (SEQ ID NO: 13).

9. The composition of claim 1, wherein the composition is combined with one or more antigens to produce a vaccine composition.

10. A method of enhancing an immune response to an antigen in a subject, the method comprising administering to a subject a vaccine composition comprising:
a. an adjuvant composition comprising an isolated peptide, the peptide comprising one or more cell-surface retention sequence (CRS) motifs, and
b. one or more antigens.

11. The method of claim 10, wherein the adjuvant composition increases the antibody titer against the antigen by about 4 to about 9 fold 14 days after vaccination, when compared to a vaccine composition that does not have the adjuvants of the invention.

12. The method of claim 10, wherein the adjuvant composition increases the antibody titer against the antigen by about >3 fold after challenge with live disease, when compared to a vaccine composition that does not have the adjuvants of the invention.

13. The method of claim 10, wherein the immune response in a subject is to a tumor-associated antigen.

14. The method of claim 10, wherein the enhanced immune response in a subject is to a pathogen antigen.

15. An isolated peptide comprising one or more cell-surface retention sequence (CRS) motif, wherein the motif is selected from the group consisting of SEQ ID NO: 1 to SEQ ID NO: 15, inclusive.

16. The peptide of claim 15, wherein the number of CRS motifs in a peptide is selected from the group consisting of 1, 2, 3, 4, and 5 CRS motifs.

17. The peptide of claim 17, wherein the CRS motifs are overlapping or contiguous.

18. The peptide of claim 15, wherein the CRS motif is selected from the group consisting of (i) a CRS motif comprising seven contiguous amino acid residues, wherein at least four of the seven amino acid residues are arginine, lysine, or histidine; (ii) a CRS motif comprising seven contiguous amino acid residues, wherein at least five of the eight amino acid residues are arginine, lysine, or histidine; (iii) a CRS motif comprising eight contiguous amino acid residues, wherein at least four of the seven amino acid residues are arginine, lysine, or histidine; and (iv) a CRS motif comprising eight contiguous amino acid residues, wherein at least five of the eight amino acid residues are arginine, lysine, or histidine.

19. The peptide of claim 15, wherein the peptide consists of a sequence selected from the group consisting of SEQ ID NO: 1 to SEQ ID NO: 15, inclusive.

20. The peptide of claim 15, wherein the peptide is selected from the group consisting of YVRVRRPPKGKHRKFKHTH (SEQ ID NO: 12) and KKSVRGKGKGQKRKRKKSRYKSWSV (SEQ ID NO: 13).


SEQUENCE LISTING (15 sequences)

SEQ ID NO: 1 - protein, 12 aa, Artificial Sequence
  SYNTHESIZED
RRRPKGRGKRRR

SEQ ID NO: 2 - protein, 18 aa, Artificial Sequence
  SYNTHESIZED
KKGFYKKKQCRPSKGRKR

SEQ ID NO: 3 - protein, 12 aa, Artificial Sequence
  SYNTHESIZED
KGRPRRGFKTRR

SEQ ID NO: 4 - protein, 8 aa, Artificial Sequence
  SYNTHESIZED
RKQRRLTR

SEQ ID NO: 5 - protein, 11 aa, Artificial Sequence
  SYNTHESIZED
KKGKKCSKTKK

SEQ ID NO: 6 - protein, 10 aa, Artificial Sequence
  SYNTHESIZED
KTGKKGKGSK

SEQ ID NO: 7 - protein, 7 aa, Artificial Sequence
  SYNTHESIZED
KTGKRKR

SEQ ID NO: 8 - protein, 9 aa, Artificial Sequence
  SYNTHESIZED
KKRKRKRLK

SEQ ID NO: 9 - protein, 7 aa, Artificial Sequence
  SYNTHESIZED
KRRGRAK

SEQ ID NO: 10 - protein, 17 aa, Artificial Sequence
  SYNTHESIZED
RTVRVRRPPKGKHRKFK

SEQ ID NO: 11 - protein, 17 aa, Artificial Sequence
  SYNTHESIZED
RTVRVRRPPKCKHRKFK

SEQ ID NO: 12 - protein, 19 aa, Artificial Sequence
  SYNTHESIZED
YVRVRRPPKGKHRKFKHTH

SEQ ID NO: 13 - protein, 25 aa, Artificial Sequence
  SYNTHESIZED
KKSVRGKGKGQKRKRKKSRYKSWSV

SEQ ID NO: 14 - protein, 19 aa, Artificial Sequence
  SYNTHESIZED
KKSVRGKGKGQKRKRKKSR

SEQ ID NO: 15 - protein, 7 aa, Artificial Sequence
  SYNTHESIZED
RQRRQKK